(19) United States
(12) Patent Application Publication
(10) Pub. No.: US 2004/0029969 A1
(43) Pub. Date: Feb. 12, 2004

(54) Use of creatine or creatine derivatives in cosmetic or dematological preparations

(75) Inventors: Thomas Blatt, Hamburg (DE); Melanie Schmidt, Hamburg (DE); Volker Schreiner, Hamburg (DE); Franz Stab, Echem (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/337,661

(22) Filed: Jan. 6, 2003

Related U.S. Application Data
(63) Continuation of application No. PCT/EP01/07510, filed on Jun. 30, 2001.

(30) Foreign Application Priority Data
Jul. 6, 2000 (DE) 100 32 964.0

(51) Int. Cl. A61K 31/198

(52) U.S. Cl. 514/565

(57) ABSTRACT

The present invention is a cosmetic or dermatological preparation or method of applying a cosmetic or dermatological preparation for the treatment or prophylaxis of skin damage or a skin condition, wherein the preparation includes creatine or a creatine derivative. The use of creatine or creatine derivatives in the cosmetic or dermatological preparations of the invention not only provides effective protection of the skin but also protects the preparations or the constituents of the preparations against harmful oxidation processes.

### CROSS-REFERENCE TO RELATED APPLICATIONS

This is a continuation application of PCT/EP01/07510, filed Jun. 30, 2001, which is incorporated herein by reference in its entirety, and also claims the benefit of German Priority Application No. 100 32 964.0, filed Jul. 6, 2000.

### FIELD OF THE INVENTION

The present invention relates to the use of creatine and/or creatine derivatives in cosmetic or dermatological preparations for the treatment and prophylaxis of the symptoms of UV- and/or ozone-induced skin damage, and also of inflammatory and degenerative skin conditions.

### BACKGROUND OF THE INVENTION

Cosmetic skin care is primarily understood as meaning that the natural function of the skin as a barrier against environmental influences (e.g. dirt, chemicals, microorganisms) and against the loss of substances intrinsic to the body (e.g. water, natural fats, electrolytes) is strengthened or restored.

Impairment of this function may lead to increased resorption of toxic or allergenic substances or to attack by microorganisms, resulting in toxic or allergic skin reactions.

Another aim of skin care is to compensate for the loss by the skin of sebum and water caused by daily washing. This is particularly important if the natural regeneration ability is inadequate. Furthermore, skincare products should protect against environmental influences, in particular against sun and wind, and delay skin aging.

Chronological skin aging is caused, for example, by endogenous, genetically determined factors. The following structural damage and functional disorders, which can also, under the term “senile xerosis”, arise, for example, in the epidermis and dermis as the result of aging:

a) Dryness, roughness and formation of dryness wrinkles,

b) Itching and

c) Reduced refatting by sebaceous glands (e.g. after washing).

Exogenous factors, such as UV light and chemical noxae, can have a cumulative effect and, for example, accelerate or supplement the endogenous aging processes. In the epidermis and dermis, for example, the following structural damage and functional disorders may arise in the skin in particular as a result of exogenous factors; these are more far-reaching than the degree and quality of the damage in the case of chronological aging:

d) Visible vascular dilation (teleangiectases, cuperosis);

e) Flaccidity and formation of wrinkles;

f) Local hyperpigmentation, hypopigmentation and abnormal pigmentation (e.g. age spots) and

g) Increased susceptibility to mechanical stress (e.g. cracking).

Products for the care of aged skin are known per se. They comprise, for example, retinoids (vitamin A acid and/or derivatives thereof) or vitamin A and/or derivatives thereof. Their effect on structural damage is, however, limited. Furthermore, in product development, there are considerable difficulties in stabilizing the active ingredients to an adequate extent against oxidative decay. The use of products containing vitamin A acid, moreover, often causes severe erythematous skin irritations. Retinoids can therefore only be used in low concentrations.

The harmful effect of the ultraviolet part of solar radiation on the skin is generally known. Whereas rays with a wavelength of less than 290 nm (the solution-called UVC region) are absorbed by the ozone layer in the earth's atmosphere, rays in the range between 290 nm and 320 nm, the so-called UVB region, cause erythema, simple sunburn or even burns of greater or lesser severity. A maximum erythema activity of sunlight is given as the relatively narrow range around 308 nm.

Numerous compounds are known for protecting against UVB radiation; these are derivatives of 3-benzylidenecamphor, of 4-aminobenzoic acid, cinnamic acid, of salicylic acid, of benzophenone and also of 2-phenylbenzimidazole.

It is also important to have available filter substances for the range between about 320 nm and about 400 nm, the so-called UVA region, since its rays can cause reactions in cases of photosensitive skin. It has been found that UVA radiation leads to damage of the elastic and collagen fibers of connective tissue, which leads to premature aging of the skin, and is to be regarded as a cause of numerous phototoxic and photoallergic reactions. The harmful effect of UVB radiation can be intensified by UVA radiation.

To protect against rays of the UVA region, therefore, certain derivatives of dibenzoylmethane are used, the photostability of which is inadequate (Int. J. Cosm. Science 10, 53 (1988)).

The UV radiation can, however, also lead to photochemical reactions, in which case the photochemical reaction products again intervene in the skin's metabolism.

Such photochemical reaction products are primarily free-radical compounds, for example hydroxyl radicals. Undefined free-radical photo products which form in the skin itself can also display uncontrolled secondary reactions due to their high reactivity. However, singlet oxygen, a non-free-radical excited state of the oxygen molecule, can also arise during UV irradiation, as can short-lived epoxides and many others. Singlet oxygen, for example, differs from triplet oxygen (free-radical ground state), which is normally present, by virtue of its increased reactivity. However, excited, reactive (free-radical) triplet states of the oxygen molecule also exist.

UV radiation is also a type of ionizing radiation. There is therefore the risk that ionic species will also form during UV exposure, which then for their part are able to intervene oxidatively in the biochemical processes.

In order to prevent these reactions, additional antioxidants and/or free-radical scavengers can be incorporated into the cosmetic or dermatological formulations.

Antioxidants are mainly used as substances which protect against the deterioration of the preparations in which they are present. Nevertheless, it is known that in human or animal skin as well, undesired oxidation processes may occur.

The essay “Skin diseases associated with oxidative injury” in “Oxidative stress in dermatology”, p. 323 ff. (Marcel Decker Inc., New York, Basle, Hong Kong, editor: Jürgen Fuchs, Frankfurt, and Lester Packer, Berkeley/California) discusses oxidative skin damage and its more likely causes.

Also for the reason of preventing such reactions, antioxidants and/or free-radical scavengers can be additionally incorporated into cosmetic or dermatological formulations.

Although a number of antioxidants and free-radical scavengers are known, for example, US patent specifications U.S. Pat. Nos. 4,144,325 and 4,248,861, and numerous other documents have already proposed the use of vitamin E, a substance with known antioxidative action in light protection formulations, the effect achieved nevertheless falls a long way short of the desired effect.

Therefore, there is a need in the art to provide cosmetic and dermatological preparations that overcome the shortcomings of the prior art.

### SUMMARY OF THE INVENTION

One object of the invention is to provide cosmetic, dermatological and pharmaceutical active ingredients and preparations, and light protection formulations which serve for the prophylaxis and treatment of photosensitive skin, in particular photodermatoses, preferably polymorphous photodermatosis. Other names for polymorphous photodermatosis are PLD, PLE, Mallorca acne and a large number of other names, as given in the literature (e.g. A. Voelckel et al, Zentralblatt Haut- und Geschlechtskrankheiten (1989), 156, p. 2).

It is also an object of the present invention to overcome the damage caused by environmental noxae and the prophylaxis in a permanent, sustained fashion without the risk of side effects.

It has surprisingly been found that the use of creatine and/or creatine derivatives in cosmetic or dermatological preparations for the treatment and prophylaxis of the symptoms of UV- and/or ozone-induced skin damage and of inflammatory and degenerative skin conditions overcomes the disadvantages of the prior art.

The present invention relates to products that use creatine and/or creatine derivatives for the care of skin stressed by the environmental noxae, such as, for example, UV light, ozone, cigarette smoke, and also for the treatment of the damage caused by photoaging, in particular of the phenomena listed under a) to g) above.

The present invention also relates to cosmetic preparations that not only provide effective protection against harmful oxidation processes in the skin, but also that protect the cosmetic preparations themselves and/or the constituents of cosmetic preparations against harmful oxidation processes.

### DETAILED DESCRIPTION OF THE PREFERRED EMBODIMENTS

Creatine (from the Greek: τοκρεασ=“the meat”) is characterized by the following structure:

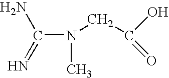

It is found in the myoserum of vertebrates in amounts of 0.05-0.4%, in small amounts also in the brain and blood. Being a monohydrate, it is a colorless, crystalline powder. In aqueous solution, creatinine is formed. In the organism, it is formed by the transamidination of L-arginine on glycine to give guanidinoacetic acid and subsequent methylation thereof by means of S-adenosylmethionine (by guanidinoacetate methyltransferase). The creatine is regarded as an appetite-promoting constituent of beef and meat extract. The addition of creatine to the diet enhances physical performance.

Cosmetic or dermatological preparations according to the invention preferably comprise 0.001-10% by weight, particularly preferably 0.01-1% by weight, of creatine and/or creatine derivative, based on the overall composition of the preparations.

A preferred derivative is creatine phosphate, which has the following structure:

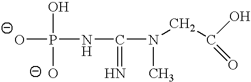

and which is distributed within fresh muscle, where it plays an important role as an energy-storing phosphate (phosphagen). In the working muscle, creatine phosphate gives, with adenosine 5′-diphosphate under the influence of the enzyme creatine kinase, adenosine 5′-triphosphate (ATP) and creatine; in the static muscle, the reverse reaction proceeds.

However, creatine sulfate, creatine acetate, creatine ascorbate and the derivatives esterified on the carboxyl group with mono- or polyfunctional alcohols also lead to advantageous embodiments of the invention.

The use of the active ingredient used according to the invention or cosmetic or topical dermatological preparations with an effective content of active ingredient used according to the invention surprisingly enables effective treatment, but also prophylaxis

of deficient, sensitive or hypoactive skin states or deficient, sensitive or hypoactive states of skin appendages

of certain degenerative symptoms of the skin (e.g. skin sagging, age spots, teleangiectases and decrease in the epidermal and dermal cell layers, the constituents of connective tissue, the retinal cones and capillary vessels of the skin) and/or of the skin appendages,

of environmentally induced (smoke, smog, reactive oxygen species, free radicals) and, in particular, light-induced negative changes in the skin and the skin appendages.

of light-induced skin damage

of pigmentation disorders,

of itching,

of horny layer barrier disorders,

of hair loss and for improved hair growth

of inflammatory skin conditions and also atopic eczema, seborrheic eczema, polymorphous photodermatosis, psoriasis, vitiligo.

The active ingredient according to the invention or cosmetic or topical dermatological preparations with an effective content of active ingredient according to the invention, however, surprisingly serves

to calm sensitive or irritated skin

to stimulate the synthesis of collagen, hyaluronic acid and elastin

to stimulate intracellular DNA synthesis, in particular in cases of deficient or hypoactive skin states

to increase cell renewal and regeneration of the skin

to increase the skin's own protective and repair mechanisms (for example for dysfunctional enzymes, DNA, lipids, proteins)

for the pre- and post-treatment in cases of topical application of laser and abrasive treatments, which serve, for example, to reduce skin wrinkles and scars, to counteract the resulting skin irritations and to promote the regeneration processes in the damaged skin.

In particular, according to the invention, it is extremely advantageous to use the active ingredient used according to the invention or cosmetic or topical dermatological preparations with an effective content of active ingredient used according to the invention for the cosmetic or dermatological treatment or prophylaxis of undesired skin conditions.

According to the invention, customary antioxidants can be added to preparations which comprise the active ingredient combinations according to the invention.

The antioxidants are advantageously chosen from the group consisting of amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example buthionine-sulfoximines, homocysteine-sulfoximine, buthionine sulfones, penta-, hexa- and heptathionine-sulfoximine) in very low tolerated doses (for example pmol to μmol/kg), and furthermore (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, alaninediacetic acid, flavanoids, polyphenols, catechins, vitamin C and derivatives (for example ascorbyl palmitate, Mg ascorbyl phosphate, ascorbyl acetate), tocopherols and derivatives (for example vitamin E acetate), and coniferyl benzoate of benzoin resin, rutic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example selenium methionine), stilbenes and derivatives thereof (for example stilbene oxide, transstilbene oxide) and the derivatives of these active ingredients mentioned which are suitable according to the invention (salts, esters, ethers, sugars, nucleotides, nucleosides, peptides and lipids).

The amount of the abovementioned antioxidants (one or more compounds) in the preparations is preferably from 0.001 to 30% by weight, particularly preferably 0.05-20% by weight, in particular 1-10% by weight, based on the total weight of the preparation.

The prophylaxis or the cosmetic or dermatological treatment with the active ingredient used according to the invention or with the cosmetic or topical dermatological preparations having an effective content of active ingredient used according to the invention is carried out in the usual manner, namely by applying the active ingredient used according to the invention or the cosmetic or topical dermatological preparations having an effective content of active ingredient used according to the invention to the affected areas of skin.

The active ingredient used according to the invention can advantageously be incorporated into customary cosmetic and dermatological preparations which may be in a variety of forms. They can, for example, be a solution, an emulsion of the water-in-oil (W/O) type or of the oil-in-water (O/W) type, or a multiple emulsion, for example of the water-in-oil-in-water (W/O/W) type or oil-in-water-in-oil (O/W/O) type, a hydrodispersion or lipodispersion, a gel, a solid stick or an aerosol.

Emulsions according to the invention for the purposes of the present invention, e.g. in the form of a cream, a lotion, a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active ingredient used according to the invention into aqueous systems or surfactant preparations for cleansing the skin and the hair.

The person skilled in the art is of course aware that demanding cosmetic compositions are mostly inconceivable without customary auxiliaries and additives. Examples thereof include bodying agents, fillers, perfume, dyes, emulsifiers, additional active ingredients, such as vitamins or proteins, light protection agents, stabilizers, insect repellents, alcohol, water, salts, and antimicrobially, proteolytically or keratolytically active substances etc.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g. Cosmetics Directive, Foods and Drugs Act).

In this connection, it is likewise advantageous to add the active ingredient used according to the invention as an additive to preparations which already comprise other active ingredients for other purposes.

Accordingly, for the purposes of the present invention, cosmetic or topical dermatological compositions can, depending on their formulation, be used, for example, as skin protection cream, cleansing milk, sunscreen lotion, nourishing cream, day or night cream, etc. In some instances it is possible and advantageous to use the compositions according to the invention as bases for pharmaceutical formulations.

Also favourable in some instances are cosmetic and dermatological preparations which are in the form of a sunscreen. As well as the active ingredient used according to the invention, these preferably additionally comprise at least one UVA filter substance and/or at least one UVB filter substance and/or at least one inorganic pigment.

It is, however, also advantageous for the purposes of the present invention to provide cosmetic and dermatological preparations whose main purpose is not protection against sunlight, but which nevertheless have a content of UV protection substances. Thus, for example, UV-A and/or UV-B filter substances are usually incorporated into day creams.

Preparations according to the invention can advantageously comprise substances which absorb UV radiation in the UVB region, the total amount of filter substances being, for example, 0.1% by weight to 30% by weight, preferably 0.5 to 10% by weight, in particular 1 to 6% by weight, based on the total weight of the preparations.

The UVB filters can be oil-soluble or water-soluble. Examples of oil-soluble substances are:

3-benzylidenecamphor and derivatives thereof, e.g. 3-(4-methylbenzylidene)camphor,

4-aminobenzoic acid derivatives, preferably 2-ethylhexyl 4-(dimethylamino)benzoate, amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy4-methoxy-4′-methylbenzophenone, 2,2′-dihydroxy-4-methoxybenzophenone;

esters of benzalmalonic acid, preferably di(2-ethylhexyl) 4-methoxybenzalmalonate;

2,4,6-trianilino(p-carbo-2′-ethyl-1′-hexyloxy)-1,3,5-triazine.

Advantageous water-soluble substances are:

2-phenylbenzimidazole-5-sulfonic acid and salts thereof, e.g. sodium, potassium or triethanolammonium salts;

sulfonic acid derivatives of benzophenones, preferably 2-hydroxy-4-methoxybenzophenone-5-sulfonic acid and its salts;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts.

The list of said UVB filters which can be used according to the invention is of course not intended to be limiting.

The invention also provides the combination of a UVA filter according to the invention with a UVB filter or a cosmetic or dermatological preparation according to the invention which also comprises a UVB filter.

It can also be advantageous to use UVA filters which are customarily present in cosmetic and/or dermatological preparations in preparations according to the invention. Such filter substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. Preparations which comprise these combinations are also provided by the invention. It is possible to use the same amounts of UVA filter substances which have been given for UVB filter substances.

Cosmetic and/or dermatological preparations for the purposes of the present invention can also comprise inorganic pigments which are customarily used in cosmetics for protecting the skin against UV rays. These are oxides of titanium, zinc, iron, zirconium, silicon, manganese, aluminum, cerium and mixtures thereof, and modifications in which the oxides are the active agents. Particular preference is given to pigments based on titanium dioxide. It is possible to use the amounts given for the above combinations.

The cosmetic and dermatological preparations according to the invention can comprise cosmetic active ingredients, auxiliaries and/or additives as are customarily used in such preparations, e.g. antioxidizing agents, preservatives, bactericides, perfumes, antifoams, dyes, pigments which have a coloring action, thickeners, surface-active substances, emulsifiers, emollients, moisturizers and/or humectants, fats, oils, waxes or other customary constituents of a cosmetic or dermatological formulation, such as alcohols, polyols, polymers, foam stabilizers, electrolytes, organic solvents or silicone derivatives.

If the cosmetic or dermatological preparation for the purposes of the present invention is a solution or emulsion or dispersion, solvents which may be used are:

water or aqueous solutions;

oils, such as triglycerides of capric or caprylic acid, but preferably castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water can be a further constituent.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24 carbon atoms, in particular 12-18 carbon atoms. The fatty acid triglycerides can, for example, be advantageously chosen from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Particularly advantageous mixtures are those of C₁₂₋₁₅-alkyl benzoate and 2-ethylhexyl isostearate, those of C₁₂₋₁₅-alkyl benzoate and isotridecyl isononanoate, and those of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

Advantageously, the oil phase can also have a content of cyclic or linear silicone oils, or consist entirely of such oils, although it is preferred to use an additional content of other oil phase components apart from the silicone oil or the silicone oils.

Cyclomethicone (octamethylcyclotetrasiloxane) is advantageously used as the silicone oil to be used according to the invention. However, other silicone oils can also be used advantageously for the purposes of the present invention, for example hexamethylcyclotrisiloxane, polydimethylsiloxane, poly(methylphenylsiloxane).

Mixtures of cyclomethicone and isotridecyl isononanoate, and of cyclomethicone and 2-ethylhexyl isostearate are also particularly advantageous.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol and, in particular, one or more thickeners which can advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of Carbopols, for example Carbopol grades 980, 981, 1382, 2984, 5984, in each case individually or in combination.

Gels used according to the invention usually comprise alcohols of low carbon number, e.g. ethanol, isopropanol, 1,2-propanediol, glycerol and water or an abovementioned oil in the presence of a thickener which, in the case of oily alcoholic gels, is preferably silicon dioxide or an aluminum silicate, and, in the case of aqueous-alcoholic or alcoholic gels, is preferably a polyacrylate. Solid sticks comprise, for example, natural or synthetic waxes, fatty alcohols or fatty acid esters.

Customary bases which are suitable for use as cosmetic sticks for the purposes of the present invention are liquid oils (e.g. paraffin oils, castor oil, isopropyl myristate), semisolid constituents (e.g. vaseline, lanolin), solid constituents (e.g. beeswax, ceresine and microcrystalline waxes and ozokerite) and high-melting waxes (e.g. carnauba wax, candelilla wax).

Suitable propellants for cosmetic and/or dermatological preparations which can be sprayed from aerosol containers for the purposes of the present invention are the customary known readily volatile, liquefied propellants, for example hydrocarbons (propane, butane, isobutane), which can be used alone or in a mixture with one another. Compressed air can also be used advantageously.

The person skilled in the art is of course aware that there are propellants which are nontoxic per se and are in principle suitable for realizing the present invention in the form of aerosol preparations, but which must nevertheless be avoided because of their unacceptable impact on the environment or other accompanying circumstances, in particular fluorinated hydrocarbons and chlorofluorocarbons (CFCs).

For the purposes of the present invention, cosmetic preparations can also be in the form of gels which, in addition to an effective content of the active ingredient according to the invention and solvents customarily used therefor, preferably water, also comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates, such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The examples below serve to illustrate the present invention.

### EXAMPLE 1

W/O cream

| | % by wt. |
|---|---|
| Paraffin oil (DAB (German | 10.00 |
| Pharmacopeia) 9) | |
| Petrolatum | 4.00 |
| Wool wax alcohol | 1.00 |
| PEG-7 hydrogenated castor oil | 3.00 |
| Aluminum stearate | 0.40 |
| Glycerol | 2.00 |
| Preservatives, dyes, perfume | q.s. |
| Phosphocreatine | 0.20 |
| Water | ad 100.00 |

### EXAMPLE 2

W/O lotion

| | % by wt. |
|---|---|
| Paraffin oil (DAB 9) | 20.00 |
| Petrolatum | 4.00 |
| Glucose sesquiisostearate | 2.00 |
| Aluminum stearate | 0.40 |
| Phytoene | 1.00 |
| Glycerol | 5.00 |
| Preservatives, dyes, perfume | q.s. |
| Creatine | 2.50 |
| Water | ad 100.00 |

### EXAMPLE 3

O/W lotion

| | % by wt. |
|---|---|
| Paraffin oil (DAB 9) | 8.00 |
| Isopropyl palmitate | 3.00 |
| Petrolatum | 4.00 |
| Cetylstearyl alcohol | 2.00 |
| PEG 40 castor oil | 0.50 |
| Sodium cetylstearyl sulfate | 0.50 |
| Sodium carbomer | 0.40 |
| Glycerol | 3.00 |
| α-Glucosylrutin | 0.20 |
| Octyl methoxycinnamate | 5.00 |
| Butylmethoxydibenzoylmethane | 1.00 |
| Preservatives, dyes, perfume | q.s. |
| Creatine acetate | 0.02 |
| Water | ad 100.00 |

### EXAMPLE 4

O/W cream

| | % by wt. |
|---|---|
| Paraffin oil (DAB 9) | 7.00 |
| Avocado oil | 4.00 |
| Glyceryl monostearate | 2.00 |
| Sodium lactate | 3.00 |
| Glycerol | 3.00 |
| Preservatives, dyes, perfume | q.s. |
| Phosphocreatine | 0.4 |
| Water | ad 100.00 |

### EXAMPLE 5

Liposome-containing gel

| | % by wt |
|---|---|
| Lecithin | 6.00 |
| Shea butter | 3.00 |
| Taurine | 0.20 |
| Sodium citrate | 0.50 |
| Glycine | 0.20 |
| Urea | 0.20 |
| Sodium PCA | 0.50 |
| Hydrolyzed collagen | 2.00 |
| Xanthan gum | 1.40 |
| Sorbitol | 3.00 |
| Preservatives, dyes, perfume | q.s. |
| Creatine | 1.20 |
| Water | ad 100.00 |

### EXAMPLE 6

Sunscreen emulsion

| | % by wt. |
|---|---|
| Cyclomethicone | 2.00 |
| Cetyldimethicone copolyol | 0.20 |
| PEG-22 dodecyl copolymer | 3.00 |
| Paraffin oil (DAB 9) | 2.00 |
| Caprylic/capric triglyceride | 5.80 |
| Octyl methoxycinnamate | 5.80 |
| Butylmethoxydibenzoylmethane | 4.00 |
| Phospocreatine | 0.50 |
| D-Biotin | 0.50 |
| ZnSO₄ | 0.70 |
| Na₄EDTA | 0.30 |
| Preservatives, dyes, perfume | q.s. |
| Water | ad 100.00 |

### EXAMPLE 7

Sunscreen emulsion

| | % by wt. |
|---|---|
| Cyclomethicone | 2.00 |
| Cetylstearyl alcohol + PEG-40 hydrogenated | 2.50 |
| castor oil + sodium cetylstearyl sulfate | |
| Glyceryl lanolate | 1.00 |
| Capryl/capric triglyceride | 0.10 |
| Laurylmethicone copolyol | 2.00 |
| Octyl stearate | 3.00 |
| Castor oil | 4.00 |
| Glycerol | 3.00 |
| Acrylamide/sodium acrylate copolymer | 0.30 |
| Hydroxypropylmethylcellulose | 0.30 |
| Octyl methoxycinnamate | 5.00 |
| Butylmethoxydibenzoylmethane | 0.50 |
| Creatine | 0.20 |
| α-Tocopheryl acetate | 1.00 |
| Na₃HEDTA | 1.50 |
| Preservatives, dyes, perfume | q.s. |
| Water | ad 100.00 |

### EXAMPLE 8

Sunscreen emulsion

| | % by wt. |
|---|---|
| Cyclomethicone | 2.00 |
| Cetylstearyl alcohol + PEG-40 hydrogenated | 2.50 |
| castor oil + sodium cetylstearyl sulfate | |
| Glyceryl lanolate | 1.00 |
| Capryl/capric triglyceride | 0.10 |
| Laurylmethicone copolyol | 2.00 |
| Octyl stearate | 3.00 |
| Castor oil | 4.00 |
| Glycerol | 3.00 |
| Acrylamide/sodium acrylate copolymer | 0.30 |
| Hydroxypropylmethylcellulose | 0.30 |
| Octyl methoxycinnamate | 5.00 |
| Butylmethoxydibenzoylmethane | 0.75 |
| Na₃HEDTA | 1.50 |
| Preservatives, dyes, perfume | q.s. |
| O-Acetyl-L-carnitine | 3.00 |
| Water | ad 100.00 |

### EXAMPLE 9

Spray formulation

| | % by wt. |
|---|---|
| Ubiquinone 10 | 0.10 |
| Creatine | 0.80 |
| Ethanol | 28.20 |
| Preservatives, dyes, perfume | q.s. |
| Propane/butane 25/75 | ad 100.00 |

### EXAMPLE 10

O/W cream

| | % by wt. |
|---|---|
| Paraffin oil (DAB 9) | 7.00 |
| Soybean oil | 4.00 |
| Glyceryl monostearate | 2.00 |
| Sodum lactate | 3.00 |
| Glycerol | 8.00 |
| Preservatives, dyes, perfume | q.s. |
| Phosphocreatine | 0.08 |
| Water | Ad 100.00 |

That which is claimed:

1. A method for the treatment or prophylaxis of the symptoms of skin damage or a skin condition, comprising applying a cosmetic or dermatological preparation comprising one or more compounds selected from the group consisting of creatine and creatine derivatives to the skin damage or skin condition.

2. The method as claimed in claim 1, said method for the treatment or prophylaxis of one or more manifestations from the group consisting of deficient, sensitive or hypoactive skin state or deficient, sensitive or hypoactive states of skin appendages; degenerative symptoms of the skin or skin appendages; environmentally induced or light-induced negative changes in the skin or the skin appendages; light-induced skin damage; pigmentation disorders; itching; horny layer barrier disorders; hair loss; inflammatory skin conditions; atopic eczema; seborrheic eczema; polymorphous photodermatosis; psoriasis; vitiligo; sensitive or irritated skin; deficiencies in the synthesis of collagen, hyaluronic acid and elastin; and deficiencies in intracellular DNA synthesis.

3. The method as claimed in claim 2, said method for the treatment or prophylaxis of polymorphous photodermatosis.

4. The method as claimed in claim 2, said method for the treatment or prophylaxis of environmentally-induced negative changes to the skin and the skin appendages caused by smoke, smog, reactive oxygen species and free radicals.

5. The method as claimed in claim 2, said method for the treatment or prophylaxis of itching.

6. The method as claimed in claim 2, said method for the treatment or prophylaxis of hair loss.

7. The method as claimed in claim 2, said method for the treatment or prophylaxis of deficiencies in the synthesis of one or more of collagen, hyaluronic acid and elastin.

8. The method as claimed in claim 2, said method for the treatment or prophylaxis of deficiencies in intracellular DNA synthesis.

9. The method as claimed in claim 2, said method for the treatment or prophylaxis of vitiligo.

10. The method as claimed in claim 1, wherein the preparation comprises a creatine derivative selected from the group consisting of creatine phosphate, creatine sulfate, creatine acetate, creatine ascorbate and esters of creatine and mono- or polyfunctional alcohols.

11. The method as claimed in claim 10, wherein the creatine derivative is creatine phosphate.

12. The method as claimed in claim 1, wherein the one or more compounds in the preparation selected from the group consisting of creatine and creatine derivatives are present in an amount from 0.001-0.49% by weight, based on the total weight of the preparation.

13. The method as claimed in claim 1, wherein the one or more compounds in the preparation selected from the group consisting of creatine and creatine derivatives are present in an amount from 10.1-30% by weight, based on the total weight of the preparation.

14. The method as claimed in claim 1, wherein the preparation further comprises one or more natural active ingredients and derivatives thereof selected from the group consisting of alpha-lipoic acid, phytoene, D-biotin, coenzyme Q10, alpha-glucosylrutin, carnitine, carnosine, isoflavone and taurine.

15. A cosmetic or dermatological preparation comprising one or more compounds selected from the group consisting of creatine and creatine derivatives for the treatment or prophylaxis of skin damage or a skin condition.

16. The preparation as claimed in claim 15, comprising a creatine derivative wherein the creatine derivative is selected from the group consisting of creatine phosphate, creatine sulfate, creatine acetate, creatine ascorbate and esters of creatine and mono- or polyfunctional alcohols.

17. The preparation as claimed in claim 16, wherein the creatine derivative is creatine phosphate.

18. The preparation as claimed in claim 15, wherein one or more compounds selected from the group consisting of creatine and creatine derivatives are present in an amount from 0.001-0.49% by weight, based on the total weight of the preparation.

19. The preparation as claimed in claim 15, wherein one or more compounds selected from the group consisting of creatine and creatine derivatives are present in an amount from 10.1-30% by weight, based on the total weight of the preparation.

20. The preparation as claimed in claim 15, wherein the preparation further comprises one or more natural active ingredients and derivatives thereof selected from the group consisting of alpha-lipoic acid, phytoene, D-biotin, coenzyme Q10, alpha-glucosylrutin, carnitine, carnosine, isoflavone and taurine.
